(19) United States
(12) Patent Application Publication
(10) Pub. No.: US 2015/0173911 A1
(43) Pub. Date: Jun. 25, 2015

(54) SHOCK ABSORBING, TOTAL DISC REPLACEMENT PROSTHETIC

(71) Applicant: TRUEMOTION SPINE, INC., GAINESVILLE, FL (US)

(72) Inventor: KEITH L. DOTY, GAINESVILLE, FL (US)

(21) Appl. No.: 14/589,435

(22) Filed: Jan. 5, 2015

Related U.S. Application Data
(63) Continuation of application No. PCT/US2013/049262, filed on Jul. 3, 2013.
(60) Provisional application No. 61/668,536, filed on Jul. 6, 2012.

(51) Int. Cl. A61F 2/44

(52) U.S. Cl. CPC A61F 2/442; A61F 2002/4435

(57) ABSTRACT

Devices for implantation within a Functional Spinal Unit (FSU) that can provide up to six independent degrees of freedom from a neutral position are provided. Such devices can comprise two endplates, a nucleus, a sled, and an optional, elastic boot attached to the endplates and enclosing the contents of the device, namely, the nucleus. The sled on one end of the nucleus can be moveably attached to one of the endplates and a spherical cap affixed to the opposite end of the nucleus can be operably engaged with the other endplate. The endplates can provide outer surface features that allow fusion of the plates to the superior and inferior vertebrae of a FSU and can prevent expulsion of the device immediately after implanting. The nucleus can have compliant elements or can be rigid. It can comprise a tripartite construction of three elements of possibly different materials that can be fitted together, or it can be a single unified element or can be a material with graded mechanical moduli axially and radially. The endplates and nucleus can maintain connection through the sled and the extension of the central core. Additionally, one or more specialized rotational joint stops can be utilized to provide profile-closure of the spherical joint between the top endplate and the spherical cap.

### CROSS-REFERENCE TO RELATED APPLICATIONS

This application is a continuation of International Patent Application No. PCT/US2013/049262, filed Jul. 3, 2013, which claims the benefit of U.S. Provisional Application Ser. No. 61/668,536, filed Jul. 6, 2012, which is hereby incorporated by reference herein in its entirety, including any figures, tables, or drawings.

### BACKGROUND OF INVENTION

Spinal disc herniation, a common ailment, and degenerative disc disease often induce pain, as well as neurologically and physiologically debilitating consequences, for which relief becomes paramount. If conservative treatments fail, the more drastic measures of discectomies and spinal fusion may be indicated. The latter treatment, while providing short term relief, limits spinal mobility and often leads to excessive forces on facet joints adjacent to the fusion and may create further problems over time. Drastic treatments are usually unable to restore normal disc function. The loss of disc function has led to a number of disc prostheses that attempt to provide natural motion.

The literature documents that the Instantaneous Axis of Rotation (JAR) during sagittal rotation of the superior vertebra with respect to the inferior vertebra of a Functional Spinal Unit (FSU) in the cervical spine moves significant distances during flexion and extension of the spine (Mameren H. van, Sanches H., Beursgens J., Drukker, J., “Cervical Spine Motion in the Sagittal Plane II: Position of Segmental Averaged Instantaneous Centers of Rotation-A Cineradiographic Study”, Spine 1992, Vol. 17, No. 5, pp. 467-474). This motion can vary widely between functional spinal units on an individual spine and between individuals and can depend on age, time of day, and the general health and condition of the intervertebral discs, facet joints, and other components of the FSU and spine. A moving IAR means that the superior vertebra can both rotate and translate while moving with respect to the inferior vertebra of an FSU.

Researchers have attempted to design a successful intervertebral disc for years. See, for example, the devices described by Salib et al., U.S. Pat. No. 5,258,031; Marnay, U.S. Pat. No. 5,314,477; Boyd et al., U.S. Pat. No. 5,425,773; Yuan et al., U.S. Pat. No. 5,676,701; and Larsen et al., U.S. Pat. No. 5,782,832, which describe designs that limit motion to rotation about a ball and socket when two plates are in contact. As the literature points out (Bogduk N. and Mercer S., “Biomechanics of the cervical spine. I: Normal kinematics”, Clinical Biomechanics, Elsevier, 15(2000) 633-648; and Mameren H. van, Sanches H., Beursgens J., Drukker, J., “Cervical Spine Motion in the Sagittal Plane II: Position of Segmental Averaged Instantaneous Centers of Rotation-A Cineradiographic Study”, Spine 1992, Vol. 17, No. 5, pp. 467-474), devices that restrict motion in such a manner do not allow the natural motion of the vertebrae, either for sagittal plane motion or for combined sagittal, lateral and axial motion. Further, when the two plates, as described in the cited patents, are not in contact, the devices are unable to provide stability to the intervertebral interface, which can allow free motion and lead to disc related spondylolisthesis, FSU instability and excessive facet loading.

Advances in disc arthroplasty design have resulted from intense patent activity in this area. Nonetheless, motion coupling between flexion-extension, axial rotation, and lateral bending and other functional spinal units involved in the overall spinal motion, increases the complexity and difficulty in developing a prosthetic disc replacement that realizes natural spinal motion. Producing such complex disc motions with strictly articulating joints is at best problematic.

Additional motion complexity required of a spinal disc derives from motion constraints dictated by facet joints. Complex facet joint surfaces in an FSU can significantly influence and constrain sagittal, lateral and axial motions. The orientation of these facet surfaces varies with FSU location in the spine and induces wide variations in motion parameters and constraints. The complex motion of a superior vertebra with respect to the associated inferior vertebra of an intact spinal joint segment, certainly in the cervical spine, cannot be realized by a simple rotation or simple translation, or even a combination of rotation and translation along a fixed axis, and still maintain the integrity and stability of the spinal segment, in particular the facet joints.

Natural spinal motions, therefore, can place severe requirements on the design of a prosthetic disc, requiring a minimum of six independent degrees of freedom to fully enable complete spinal motion. The number of independent mechanical degrees of freedom of a disc modeled on rigid body assumptions, however, can be reduced by one or more degrees of freedom at a joint stop or singularities of configuration, limiting further motion along the reduced degrees of freedom. Using only mechanically articulating joints, therefore, can be problematic in resolving complex disc motion. Adding flexure motion capability between relatively rigid mechanical joints within a disc prosthesis can enhance the prosthesis responsiveness when in configurations that restrict certain motions of mechanical articulating joints. On the other hand, mechanical articulating joints can provide structurally stronger and more robust motion generation with less stress. A combination of articulating joints and flexure motion structures, therefore, can enhance performance by utilizing the features of both. Such a combination of mechanical and flexural joints would represent a hybrid disc prosthetic system, hybrid because it employs both (approximately) rigid-body articulating joints and flexure links between those joints. A device utilizing a hybrid system, being one that employs rigid-body-modeled, articulating, mechanical joints linked by a nucleus, a portion of which can be compliant, can enable a total disc replacement to perform the complex, coupled motions required of a natural disc.

### BRIEF SUMMARY

The invention instructed herein meets a variety of criteria for an artificial intervertebral disc suitable for implantation within a Functional Spinal Unit (FSU). When implanted, the invention can provide up to six independent degrees of freedom from a neutral position and enables the device to exercise complex motions dictated by spinal muscles, and motions consisting of simultaneous, compound rotations coupled with simultaneous multi-axis translations. A moving Instantaneous Axis of Rotation (JAR), typically dictated by flexion and extension in the cervical spine, for example, can be successfully tracked by the embodiments of the subject invention utilizing a movable rotation center and appropriate multi-axis translations. Shock absorption and flexure along and about multiple axes are other features that can be utilized with the devices of the subject invention.

In one embodiment, the devices comprise two endplates, a nucleus, and an optional, elastic boot attached to the endplates and enclosing the contents of the device, namely, the nucleus

The endplates can provide outer surface features that allow fusion of the plates to the superior and inferior vertebrae of a FSU and can prevent expulsion of the invention immediately after implanting. This disclosure instructs, but the invention is not limited to, several endplate geometries. The perimeter of a horizontal section of an endplate can be a general closed curve of which a circle, ellipse, and rounded-corners squares, rectangles, and trapezoids describe simple instances.

The nucleus can have compliant and rigid sections or in a particular embodiment comprise only compliant materials and, in another embodiment, only rigid materials, and yet another embodiment, a combination of compliant and rigid materials.

In specific embodiments, a nucleus can be configured as an integrated, graded polymer or as a tripartite construction comprising two or more, usually three, elements that can be overmolded, fitted or bonded together, or it can be a single unified element of the same material. One embodiment comprises a viscoelastic spherical cap, core, and sled. In one example of a graded polymer nucleus, the cap, core, and sled can consist of possibly different thermoplastics with different mechanical properties to be over-molded so as to create a single unit with five regions consisting of three primary regions, each comprising a particular material and two transitions, or interface, regions therebetween. Each transition region can comprise a mixture of the material between two different primary regions to which it interfaces. Mechanical properties of the nucleus can transition from one primary region to another primary region through an interface region. Such an interface region can vary in thickness from between approximately 0.025 mm to approximately 0.1 mm as the material transitions from one concentration of material to another. Such graded interface regions can be advantageous in handling shear stresses compared to abrupt interface transitions that occur when the three elements comprise separate parts to be fitted together.

A continuously graded nucleus can be generated by varying the mechanical moduli both axial and radially using additive manufacturing techniques with viscoelastic materials.

The mechanical properties chosen for each of the three regions can dictate the choice of polymer or other material for each element, which do not have to be different. For one embodiment, all three elements (cap, core, and sled) consist of the same polymer; for another embodiment, the same polymer is used for all three regions (single unit). Variations of both types of embodiments are instructed herein. This disclosure instructs, but the invention is not limited to, several different embodiments of a nucleus, all of which can be constructed from one to three different polymers that can be joined together by one or more combinations of techniques, for example, possible combinations of overmolding, chemical bonding, thermal or sonic welding, or mechanical bonding.

In particular embodiments, the invention employs, but is not limited to, a disc shaped sled and a rounded-corners square sled. Other embodiments of sled perimeter geometries can include any closed curve, such as, for example, an ellipse. From the perimeter geometries of a sled and its corresponding bottom endplate cavity, the associated range of motion of the sled within the bottom endplate cavity can be determined by one skilled in the art. Such variations, which provide the same function, in substantially the same way, with substantially the same result, are within the scope of the subject invention.

The geometry of the sled within a bottom endplate cavity, together, determines the freedoms and range of motion. For example, a disc shaped sled with radius r within a circular cavity with radius r+δ can allow the disc to move from the center of the cavity δ mm in any direction, realizing two degrees of freedom in polar coordinates. A square sled s mm on a side within a bottom endplate cavity s+δ can allow the sled centroid to move from the cavity centroid δ mm in either the y or z direction independently (refer to FIG. 2 for coordinate frame description).

While the nucleus of the invention can be embodied using several different means of manufacture, the abstract functional, or operational characteristics, can be described for all versions without differentiation. The nucleus maintains the intervertebral space, provides two articulating mechanical joint interfaces, one with each endplate; can provide shock absorption or not, depending upon an embodiment selected; and can allow translation of the Center-of-Rotation (COR) of the spherical cap. Movement of the COR of the spherical cap, can help accommodate variations in placement of the device at implant and variations in the location of the Center-of-Rotation (COR) of the top endplate with respect to the bottom endplate from level-to-level in the cervical spine.

When two surfaces can move relative to each other to form a lower pair joint, but no externally applied forces are necessary to maintain contact between the surfaces, then the joint is said to be profile-closed. A series of mechanically linked, profile-closed joints comprises a kinematic chain.

In one embodiment, a spherical cap region at the top of the nucleus interfaces with a conforming spherical cavity in a top endplate to form a three rotational degrees of freedom “ball-and-socket” type joint. These interfacing spherical surfaces define a lower pair spherical joint. This spherical joint is not shown profile-closed in the figures, but it can be made so as discussed below.

Moving caudally, the spherical cap material can transition to a region, called the core, consisting of the same, or different, material as the spherical cap. The core can transition to a region, called a sled, consisting of the same or different, material as the core or cap. When joined together the spherical cap, core, and sled comprise the nucleus of the device. The sled can transport the rest of the nucleus and top endplate and establishes a planar joint with the bottom endplate in which the sled can move. Advantageously, this arrangement can provide up to two independent translational degrees of freedom and one independent axial rotation, which can be a redundant rotation since the spherical cap can also provide axial rotation. Together the spherical ball and socket joint and the planar joint provide 6-degrees-of-freedom (6-DOF) of which 5-DOF are independent.

In an embodiment where a portion of the nucleus is compliant, the nucleus allows a continuum number of possible motion modalities, an important one of which is compression along the y-axis (refer to FIG. 2). The latter motion generates an independent translation motion along the y-axis of the device (refer to FIG. 2) of the top endplate with respect to the bottom endplate, yielding, among other mobilities, a sixth independent degree of freedom

For a graded polymer nucleus embodiment, the transition from cap to core to sloican be gradual, as in, for example, an overmolded case. For tripartite construction, the transition can be more abrupt. A single polymer nucleus can have no material transition regions. A nucleus with compliant portion(s) can provide shock absorption for axial compression up to approximately 1 mm, which can provide a third, independent translation degree of freedom, as mentioned previously. In a particular embodiment, the translation due to the compression of compliant portions of the nucleus and the motion of the sled can provide three, mutually orthogonal, i.e. independent, translational degrees of freedom. These degrees of freedom, coupled with the three rotational degrees of freedom of a spherical cap can complete the realization of six independent degrees of freedom of motion of the top endplate with respect to the bottom endplate.

In addition to providing 6-DOF, 5 independent DOF and one redundant DOF by means of two articulating mechanical joints, the compliant portions of the nucleus can provide a continuum of redundant freedom by means of flexion, extension and torsion about any axis. This redundancy allows accommodating complex, coupled joint motions of normal spinal motion dictated by spinal muscles and tendons, even when joint limits of the articulating mechanical joints have been reached.

Particular embodiments of the viscoelastic core can also support a plurality of hydraulic circuits that assist in shock absorption and can provide synovial joint type operation by forcing fluids between the sliding and rotating surfaces. In a particular embodiment, a hydraulic fluid can be a sterile, normal saline solution encapsulated by a boot, which can isolate the core and other working components from environment or biological fluids. In an alternative embodiment, there is no boot and the hydraulic fluids circulated by the device are environmental/biological fluids. Contraction and expansion of the central hydraulic cavity and transport channels of the viscoelastic core, as it compresses and then relaxes, can supply the pumping action of the fluid. In one embodiment, duck bill valves, or other valving techniques, located at the openings of hydraulic channels within a central hydraulic chamber, can allow fluid to flow into the chamber, but not out of the chamber. Duck bill valves, or other valving techniques, can also establish one-way flow out of the central chamber through its top and bottom portals. Such a configuration, during operation, can supply the “synovial” fluid-like action for the spherical and planar joints, respectively, reducing wear and friction.

In one embodiment, progressing still further caudally, the viscoelastic core transitions to a region designated as a sled. The most caudal end or underneath surface of the sled can mate with a planar surface within a cavity in the bottom endplate to form a planar joint. For a disc shaped sled, the sled can slide up to approximately ±1 mm in any direction from the geometric centroid, or from the vertical axis, of the cavity plane. A rounded-corners square sled 2-2-3, can slide up to approximately ±1 mm in the z direction and ±1 mm in the x direction from the centroid, or from the vertical axis, of the cavity plane, where z and x define the plane of motion (refer to FIG. 2 for coordinate frame description). Other sled and bottom endplate cavity geometries that generate other motion capabilities can easily be devised, based on this instruction, by one skilled in the art.

In one embodiment of a disc sled, the orifice in the bottom endplate cavity provides a tight fit for insertion. Once inserted, a cavity lip can prevent the sled and nucleus from being expelled from between the plates during nominal operation, thus, creating a profile-closed planar joint. In an alternative embodiment of a rounded-corners square sled, a separate sled retaining ring, in lieu of a cavity lip, can be fixedly or removeably attached to the bottom endplate, after the sled has been inserted in the bottom endplate cavity. In a further embodiment, the bottom end plate cavity can be made larger than the sled dimensions to easily accommodate such a placement. After fixing the sled retaining ring to the bottom endplate, there will be a retaining lip around the cavity, to retain the sled, hence the entire nucleus, between the plates, even during motion of the sled within the cavity, thus, creating a profile-closed planar joint.

In one embodiment, a viscoelastic boot in slight tension, when fixed to the endplates, separates the invention's interior space from its external environment and can provide resistance to extension forces and general torsion. Such a boot also can protect the mechanism from osteoblasts and other cell matter from fouling the mechanism during the healing process.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. It should also be understood that the drawings presented herein may not be drawn to scale and that any reference to, or implication of, dimensions in the drawings or the following description are specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which

FIG. 1 shows a top, side perspective view of a cylindrical embodiment of the subject invention.

FIG. 2 shows a sagittal plane cross-sectional view of a cylindrical embodiment of the subject invention.

FIG. 3 shows a sagittal plane cross-sectional view of an alternative cylindrical embodiment.

FIG. 4 shows an exploded view of an embodiment of the subject invention.

FIG. 5 shows an exploded, sagittal plane cross-sectional view of an embodiment of the subject invention.

FIG. 6 shows a cross-sectional perspective view of an embodiment of a nucleus of the subject invention.

FIG. 7 shows a cross-sectional perspective view of an alternative embodiment of a nucleus of the subject invention.

FIG. 8 shows an exploded view of another alternative embodiment of a nucleus of the subject invention.

FIG. 9 shows a cross-sectional perspective view of another alternative embodiment of a nucleus of the subject invention.

FIG. 10 shows an embodiment of the invention with a rounded-corner perimeter.

FIG. 11 shows an embodiment of the invention with a specific nucleus and sled configuration.

FIG. 12 shows an embodiment of the invention with an alternative specific nucleus and sled configuration.

FIG. 13 shows an exploded perspective view of a specific embodiment of the subject invention.

FIG. 14 shows an exploded cross-sectional bottom perspective view of a specific embodiment of the subject invention.

FIG. 15 shows a top perspective view of an embodiment of a combined nucleus and sled of the subject invention.

FIG. 16 shows a top perspective view of an alternative embodiment of a combined nucleus and sled of the subject invention.

FIG. 17 shows a cross-sectional top perspective view of an embodiment of a sled and bottom end plate of the subject invention.

### DETAILED DISCLOSURE

The subject invention pertains to embodiments of a device capable of providing motion with up to six independent degrees-of-freedom and redundant flexure degrees of freedom. Specifically, articulating mechanical joints of the invention can provide five independent degrees of freedom and one redundant axial rotation degree-of freedom. Flexure between the two mechanical joints provides an additional axial degree of freedom and allows a continuum of motions within the rated modulus of the material. In particular, flexure in the invention accommodates compression-extension motions along the axial axis of the FSU independent of the articulation joint degrees of freedom. Embodiments of the device can further simultaneously provide reaction to compressive, tension and torsion loads. More specifically, the subject invention pertains to embodiments of a device capable of approximating the potential motion between two vertebrae in a spine

The following description will disclose embodiments of the subject invention that can be useful in the medical fields encompassing spinal surgery and, in particular, to devices and methods for correcting, replacing, or approximating natural movement between two adjacent vertebrae within a Functional Spinal Unit (FSU). More specifically, the embodiments disclosed herein can be useful for the treatment and/or removal of spinal disc herniation and degenerative disc disease. However, a person with skill in the art will be able to recognize numerous other uses that would be applicable to the devices and methods of the subject invention. While the subject application describes, and many of the terms herein relate to, a use for implanting within a spine, particularly for the treatment of spinal disc herniation and degenerative disc disease, other uses and modifications thereof that will be apparent to a person with skill in the art and having benefit of the subject disclosure are contemplated to be within the scope of the present invention.

In the description that follows, a number of terms are used in relation to the spine, spinal surgery, and medical devices related thereto. In order to provide a clear and consistent understanding of the specification and claims, including the scope to be given such terms, the following definitions are provided:

In this disclosure, a Functional Spinal Unit (FSU) or spinal joint is defined herein as two successive vertebrae in a spine, a superior vertebra and an inferior vertebra, including their mutual facet joints, an intervertebral disc attached to the inferior surface of the superior vertebra and the superior surface of the inferior vertebra, and connecting ligaments. Spinal muscles attached to a spinal joint or FSU can induce motion of not only that spinal joint but also influence the motion of other spinal joints due to complex mechanical couplings between spinal joints.

All possible natural motions of a superior vertebra with respect to an inferior vertebra in a healthy spinal joint define the natural workspace of a superior vertebra with respect to an inferior vertebra of a Functional Spinal Unit (FSU). This FSU workspace model treats the vertebrae as rigid bodies, in contrast to a flexible disc, and typically varies from FSU to FSU of an individual spine, and from one individual to another, creating considerable spinal disc prosthesis design problems.

The term “patient” as used herein, describes an animal, including mammals, to which the systems and methods of the present invention can be applied. Mammalian species that can benefit from the disclosed systems and methods include, but are not limited to, apes, chimpanzees, orangutans, humans, monkeys; domesticated animals (e.g., pets) such as dogs, cats, guinea pigs, hamsters; veterinary uses for large animals such as cattle, horses, goats, sheep; and any wild animal for veterinary or tracking purposes.

From bottom endplate to top endplate, for a particular embodiment, the mechanical linkage comprises a planar pair joint linked to a spherical pair joint. A flexible, or rigid, nucleus can establish one surface of a planar pair joint at the caudal end and a spherical surface of a spherical pair joint at the cranial end, and link the two joints together. The connecting link can be compliant in part or rigid, depending upon the embodiment. The planar pair joint can be profile-closed by a snap-fit or by a retaining ring. The spherical pair joint can be profile-closed by using rotational joint stops or sliders. Rotational joint stops and sliders are taught by U.S. Pat. No. 8,277,505 (Doty), which is hereby incorporated by reference for such teachings, including any figures, tables, or drawings pertaining to such teachings. When the planar and spherical joints are both profile-closed and the nucleus is flexible, there results a hybrid kinematic chain. A hybrid kinematic chain, as defined here, comprises a kinematic chain with one or more, profile-closed, lower-order pair joints, modeled as rigid-bodies, and one or more flexible links connecting such joints.

In this disclosure, the lower pairs can be augmented by the incorporation of ball bearings, roller bearings, sliding bearings, line bearings, fixed bearings, and so forth to create higher pairs. Such modifications are taught in the inventor's U.S. Pat. Nos. 7,361,192; 7,799,080; 7,927,375; 8,226,724; and 8,277,505 provide numerous examples of higher order pair joints that can be substituted or utilized with the embodiments of the subject invention. These references are hereby incorporated, for such teachings, by reference herein, including any figures, tables, or drawings pertaining to such teachings.

The present invention is more particularly described in the following embodiments and examples that are intended to be illustrative only since numerous modifications and variations therein will be apparent to those skilled in the art. As used in the specification and in the claims, the singular for “a,” “an” and “the” include plural referents unless the context clearly dictates otherwise.

Finally, reference is made throughout the application to the “cranial end” and “caudal end.” As used herein, the cranial end 100 is that end that would typically be nearest to the head of a patient. Conversely, the caudal end 200 of the device is that end that would typically be nearest the tail end of a patient (nearest the foot end in a human patient). Similarly, caudally indicates a direction toward the “tail” and cranially a direction toward the head from a current point of reference. These references are for literary convenience and it should be understood that the embodiment of the invention can be used in any orientation within a patient.

The various embodiments disclosed herein can allow up to six independent degrees-of-freedom at the center of the Functional Spinal Unit (FSU) workspace. Those degrees of freedom continue to operate until one or more joint limits have been reached. The embodiments of the invention pertaining to approximating the potential motion between two vertebrae in an animal spine can maintain the integrity of the variable intervertebral spacing required during motion. For example, under compression, the intervertebral gap can narrow some and under extension it can widen some.

When appropriately scaled, the invention is capable of tracking workspace movements, within prescribed joint limits, for up to three independent translational and three independent rotational motions of the superior vertebra with respect to the inferior vertebra of the FSU. The flexible nucleus (2-n, n=1 through 7) can allow an infinite number of motions in response to compression, extension, shear, and torsion acting upon it. This is a principle feature of the invention, namely, the device can employ both mechanical, articulating joints and material flexure to realize any motion dictated by spinal muscles. This type of mechanical structure can be called a hybrid mechanical linkage as it includes both lower-ordered pair joints and flexure links, and, if all joints are profile-closed, a hybrid kinematic chain, as defined previously. A hybrid mechanical linkage, or hybrid kinematic chain, establishes two very different mechanisms to realize mobility, rigid-body lower-ordered pair joints and link compliance.

In a patient, the invention can serve, through appropriate mechanical programming of joint stops and scaling, as a disc prosthesis for any point along the spine. For example, in a human patient, the embodiments of the device, with appropriate joint stops and scaling can be utilized as a disc prosthesis from the cervical to the lumbar regions.

For particular embodiments of the invention, the outside perimeter defined by a planar horizontal cross section of endplates 1-1, 1-2, 3-1, 3-2, 3-3 and 3-4 can be trapezoidal, rectangular, square, circular, elliptical, or a general curvate closed curve. While these various geometries can affect the shape of the superior and inferior plates, the internal joint mechanism can typically remain the same, as the two embodiments of external geometries instructed herein, for example, 1-1, and 3-1 are circular and 1-2, 3-1, 3-2, 3-3, 3-4 are rectangular.

FSU angular and translational displacement along the various degrees of freedom, as instructed herein, typically relate to cervical spine applications of the invention in a human patient. However, it should be understood that the invention is not restricted to the cervical spine and can be scaled for larger spinal joints, for example, L4-L5 in the lumbar region.

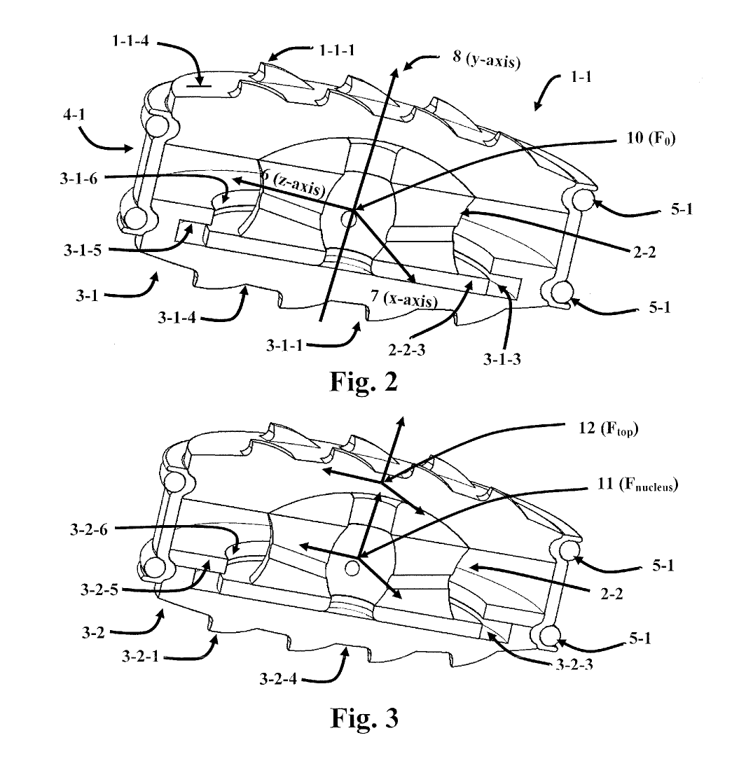

As shown in FIG. 2 a fixed reference frame F₀ refers to the right-handed coordinate system defined at the center of the device and attached to the bottom endplate, which is considered a fixed reference frame from which to describe relative motions of the nucleus and the top endplate. The labels 6, 7, and 8 designate the frontal, sagittal, and vertical plane axes of frame F₀, i.e., those axes perpendicular to their respective planes. These axes will also be referenced herein as the frontal axis, sagittal axis and the central axis. Coordinates along these axes will be designated z, x, y, respectively. Label 10 designates the coordinate frame F₀. Arrow heads on the axes point in the positive direction.

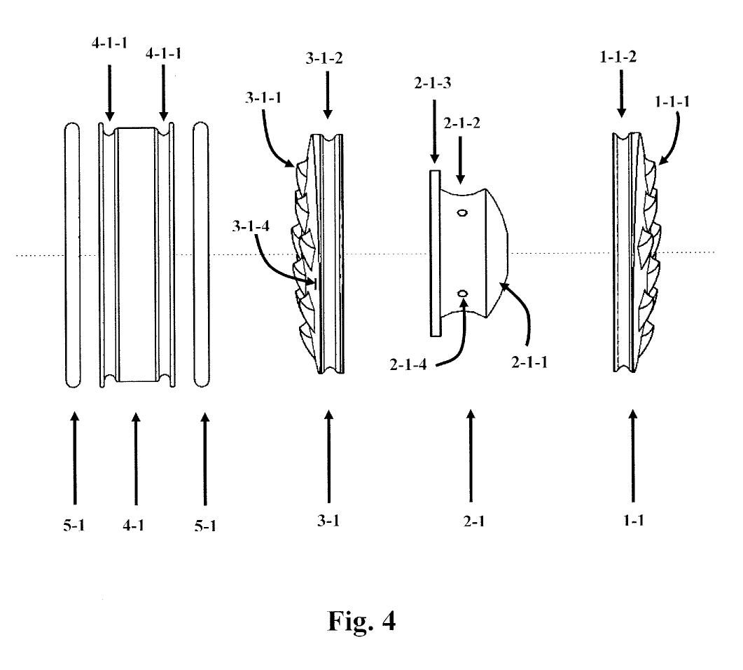

Two more right-handed-coordinate frames, shown in FIG. 3, namely, Fₜₒₚ 12 and F_{nucleus} 11, depict body attached frames for a top endplate and a nucleus. These frames move with respect to F₀ and with respect to each other. In one embodiment, F_{nucleus} can translate in three independent directions with respect to F₀. During pure compression of the viscoelastic core, F_{nucleus} can displace caudally 200 along the central axis (negative direction along y-axis) with respect to F₀. With pure extension, the viscoelastic core can expand cranially (positive direction along y-axis) along the central axis. With pure compression and extension along the central axis of the viscoelastic core presumed to be induced by a similar driving movement of the top endplate, frames F₀, Fₜₒₚ, and F_{nucleus} remain parallel to each other.

Reference will be made to the attached figures on which the same reference numerals are used throughout to indicate the same or similar components. In the attached figures, which show certain embodiments of the subject invention, it can be seen that the subject invention comprises, generally, a top endplate 1-1, a bottom end plate 3-1, and a central nucleus 2-1. Variations of these general components are disclosed and shown in the attached figures.

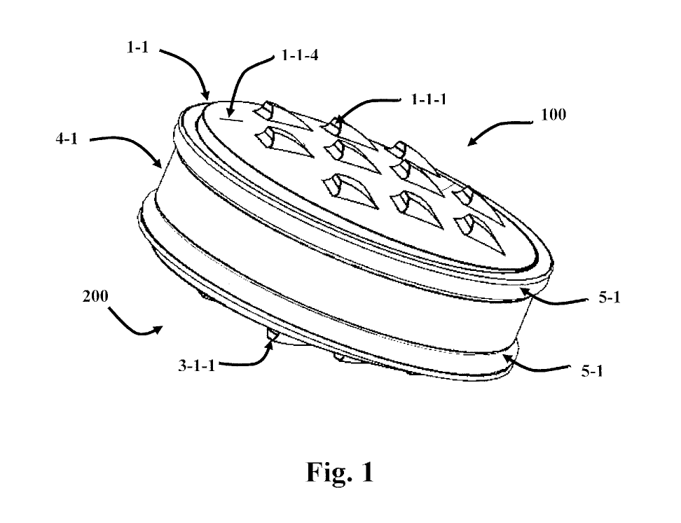

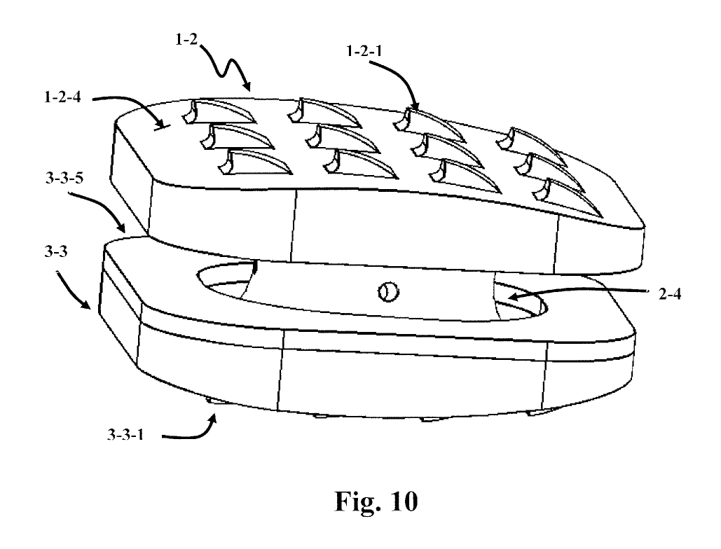

With reference to the attached figures, it can be seen that FIG. 1 depicts a cylindrical embodiment of the invention with top endplate 1-1 and protective boot 4-1 in perspective view, as seen from the front-left. In this embodiment, retention rings 5-1 hold the boot against endplates 1-1 and 3-1 (FIG. 4). Upper surface 1-1-4 of the top endplate and lower surface 3-1-4 of the bottom endplate can be treated to enhance bone ingrowth. One or more securing structures, such as, for example, cleats, chevrons, or keels 1-1-1 and 3-1-1 on the outside surfaces of the endplates can retain the endplates within the intervertebral space after implanting. The shape and size of securing structures 1-1-1 and 3-1-1 (also 3-2-1 on FIG. 3, and 3-3-1 on FIG. 10) do not have to be uniform and can be, for example, large keels or small spikes. They can also have the property of allowing easy insertion, but opposing extraction, much like a barbed arrow head.

FIG. 2 depicts a sagittal plane cutaway of the embodiment shown in FIG. 1. In this embodiment tripartite, sliding viscoelastic nucleus 2-2 can be constrained within a bottom endplate cavity 3-1-3 by a lip 3-1-5 and the protruding edges of a sled 2-2-3, which can further engage the lower surface of lip 3-1-5 when forces attempt to pull the nucleus away from the bottom endplate in the axial direction 8. In this embodiment, the sled 2-2-3 can press-fit or snap-fit through orifice 3-1-6 created by lip 3-1-5 and, in one embodiment, is free to move about the cavity floor, which lies in a plane orthogonal to axis 8. The invention body-attached-frame can comprise a right-handed coordinate system, shown in FIG. 2. The axes of this frame 10 (F₀) are designated the frontal (6, z-axis), sagittal (7, x-axis) and axial (8, y-axis) axes of the invention body and the FSU in which it is implanted. The origin of this coordinate system is centered within the device and the FSU.

FIG. 3 shows an alternative embodiment of the bottom endplate 3-2, where a separate locking element 3-2-5 retains the nucleus within cavity 3-2-3 of the bottom endplate 3-2. In this embodiment, a retaining ring 3-2-5 can fixedly attach to bottom endplate 3-2 after insertion of the nucleus. In a further embodiment, the dimensions of the sled 2-2-3 prevent it from passing through orifice 3-2-6. This embodiment, as in FIG. 2, utilizes a tripartite nucleus 2-2.

FIG. 4 depicts an exploded three dimensional view of a particular embodiment of the invention having five principal parts: a top endplate 1-1, a graded or single material viscoelastic nucleus 2-1, a bottom endplate 3-1, a boot 4-1 and a boot retaining elements 5-1. In one embodiment, endplate grooves 1-1-2 and 3-1-2 can accommodate conforming bulges on the boot, which in turn, have conforming grooves 4-1-1 that can accommodate boot retention rings 5-1.

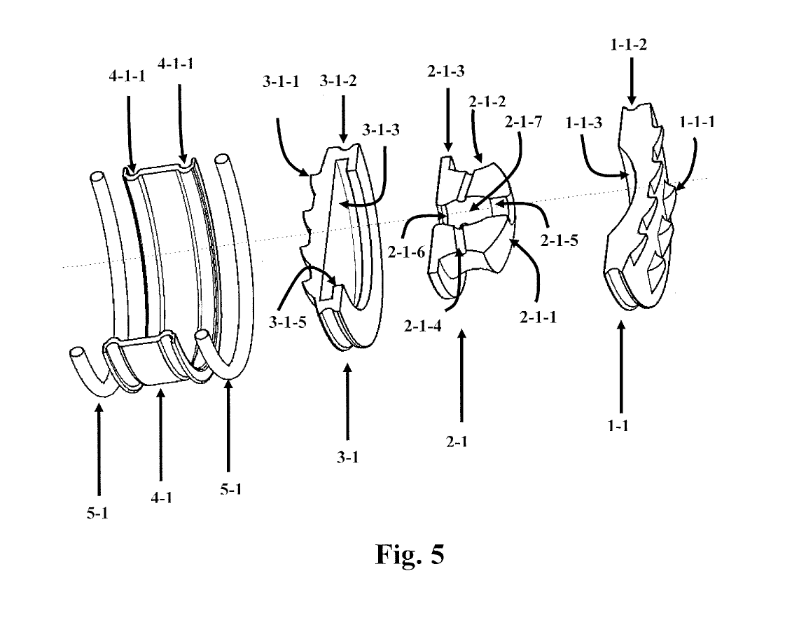

FIG. 5 shows an exploded, sagittal cutaway view of the embodiment shown in FIG. 4. This figure reveals a particular embodiment of hydraulic pathways internal to the nucleus. Bottom endplate cavity 3-1-3 with a planar joint surface can allow sled 2-1-3 to slide in the plane of the cavity. In a specific embodiment, the planar joint surface allows sled 2-1-3 to slide between approximately 0.1 mm to approximately 1 mm from the center of the cavity in any direction along the plane of the cavity. In one embodiment, a spherical cavity 1-1-3 in the top endplate conforms to the spherical surface of spherical cap 2-1-1. Lip 3-1-5 can comprise part of the structure of bottom endplate 3-1. However, as seen, for example, in FIG. 3, this lip can also be created by a sled retaining ring 3-2-5 when bottom endplate 3-2 is used instead of bottom endplate 3-1.

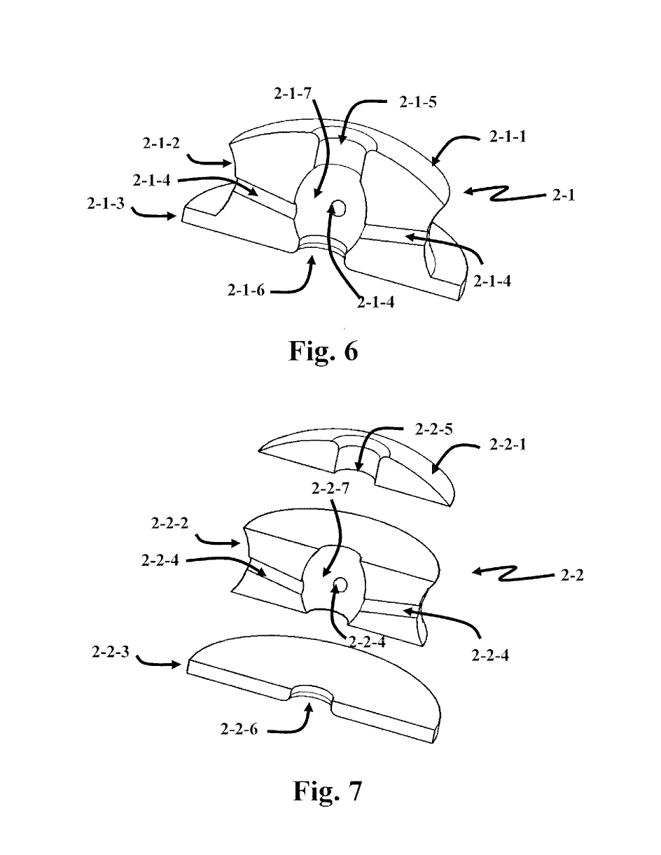

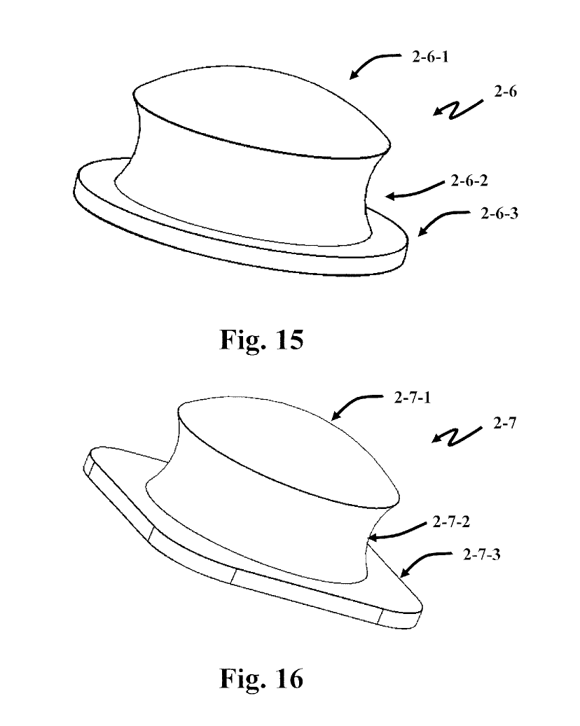

FIG. 6 shows details of an embodiment of a nucleus 2-1 with a plurality of hydraulic portals 2-1-4 and central chamber 2-1-7. In one embodiment, nucleus 2-1 is an integrated, graded polymer whose spherical cap 2-1-1 and sled 2-1-3 can have much higher durometer than the softer core 2-1-2. The three elements of this embodiment, the spherical cap 2-2-1, the core 2-2-2, and the sled 2-2-3, can be joined employing one or more means, such as, for example, overmolding, chemical bonding, mechanical bonding, thermal welding, and the like. Portals 2-1-5 and 2-1-6 allow fluid to be pumped from the central chamber 2-1-7 to lubricate both the spherical joint, formed by the spherical cap 2-1-1 and the spherical cavity 1-1-3, and the planar joint, formed by the bottom surface of sled 2-1-3 and the floor of the cavity 3-1-3. A plurality of side portals 2-1-4 can relieve chamber pressure. Duck bill valves, not shown, can be utilized with one or more portals 2-1-4 at the chamber end of the channels to allow drawing in fluids, but forcing all output fluids into the two joint systems, generally providing synovial-like action. A mix of valving techniques can create other useful hydraulic circuits to achieve different flow functions. Other embodiments can have no hydraulic circuits, portals or channels at all, for example, nuclei 2-6 and 2-7 in FIG. 15 and FIG. 16, respectively.

FIG. 7 shows details of a tripartite embodiment 2-2 of the nucleus. This embodiment consists of a spherical cap 2-2-1 and sled 2-2-3 that possess a much higher durometer than the softer viscoelastic core 2-1-2. All three of these elements can be comprised of the same or of different polymers. In one embodiment, viscoelastic core 2-2 fixes to the caudal 200 or lower surface of spherical cap 2-2-1 and the cranial 100 or upper surface of sled 2-2-3. When assembled the resultant nucleus can have a hydraulic portal structure, such as shown in FIG. 6 or none at all (2-6 and 2-7).

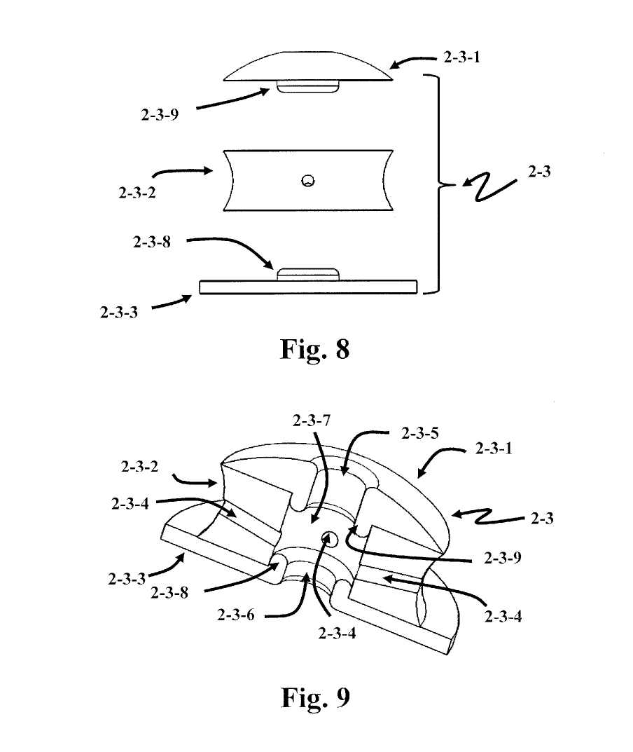

FIG. 8 indicates an alternative embodiment of a tripartite nucleus embodiment 2-2 wherein the spherical cap 2-3-1 and sled 2-3-3 possess fixed tubular projections 2-3-9 and 2-3-8, respectively, that partially extend into the hydraulic central chamber. These projections can help to mechanically support bonding between the viscoelastic core 2-3-2 with spherical cap 2-3-1 and sled 2-3-3. In one embodiment, these projections can be much stiffer or more rigid than the core material and, thus, can limit the compression of the core by interfering with each other and increasing the stiffness against further compression accordingly.

FIG. 9 illustrates a cross-section of one embodiment of a nucleus 2-3. Embodiment 2-3 has all the structural elements of 2-1 with the addition of tubular elements 2-3-8 and 2-3-9 entering the upper and lower orifices of central chamber 2-3-7. Features of the nucleus embodiment in this figure, namely, spherical cap 2-3-1, viscoelastic core 2-3-2, sled 2-3-3, hydraulic portals 2-3-4, 2-3-5, and 2-3-6, and central chamber 2-3-7, correspond to the spherical cap 2-1-1, viscoelastic core 2-1-2, sled 2-1-3, hydraulic portals 2-1-4, 2-1-5, 2-1-6, and central chamber 2-1-7 of nucleus embodiment 2-1, shown in FIG. 6.

FIG. 10 shows an embodiment of the invention without a boot and with a polyline perimeter, shown here, for example, as a rounded-corner trapezoid, square, or rectangle. The top and bottom endplates 1-2 and 3-3, respectively, can include keels, spikes, chevrons, cleats or similar fixing elements 1-2-1 and 3-3-1.

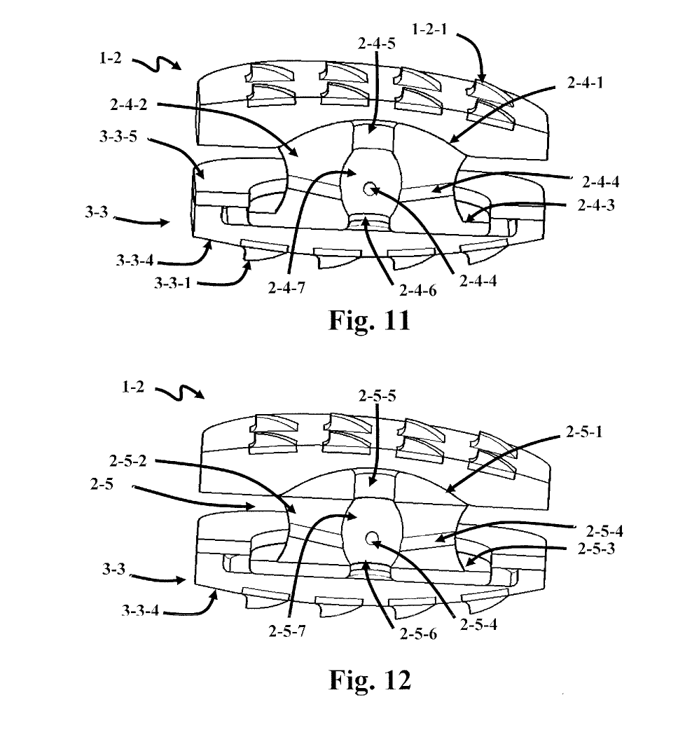

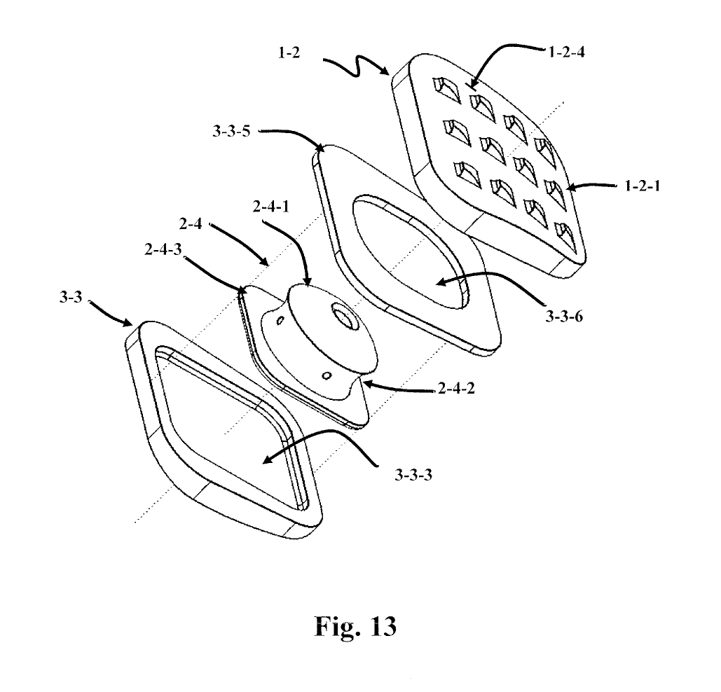

FIG. 11 shows an embodiment 2-4 of an integrated, graded polymer nucleus or one constructed from a single material. In this embodiment, the sled comprises a rounded-corner square (refer to FIG. 13) shape, as compared to the disc sled of nucleus 2-1, shown, for example, in FIG. 6. All other design features of the two nuclei embodiments can be the same; specifically, elements 2-1-n and 2-4-n can be equal where n=1, 2, 4, 5, 6, 7. Other nucleus embodiments, such as a single material constructed one, which has no hydraulic portals or channels, can be substituted for 2-4 here.

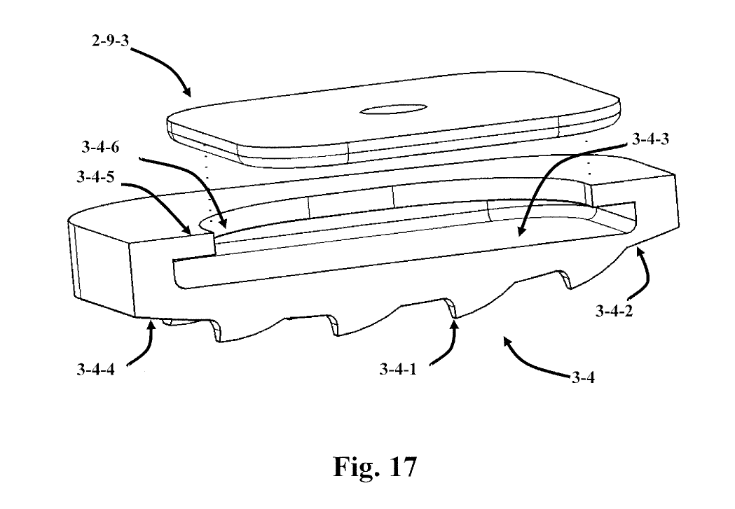

FIG. 12 shows an embodiment of a tripartite nucleus embodiment 2-5 with a rounded-corners square sled geometry 2-5-3 as opposed to a disc sled 2-2-3. Specifically, elements 2-5-n and 2-2-n can be equal where n=1, 2, 4, 5, 6, 7. Other nucleus embodiments, such as tripartite version 2-7, shown, for example, in FIG. 17, which has no hydraulic portals or channels, can be substituted for 2-5 here.

FIG. 13 illustrates an exploded view of an embodiment having the rounded corner square cavity 3-3-3 in the bottom endplate 3-3. Retention ring 3-3-5 with opening 3-3-6 can pass over cap and core of a nucleus, having a sled 2-4-3 disposed within the rounded corner square cavity 3-3-3, and be fixedly attached to the bottom endplate. In this embodiment, sled 2-4-3 cannot pass through opening 3-3-6 and, thereby, nucleus 2-4 is retained within the cavity 3-3-3. Endplate cleats, chevrons, or keels 1-2-1 and 3-3-1 can serve the same function as endplate cleats, chevrons, or keels 1-1-1 and 3-1-1, shown previously. Advantageously, any embodiment of a nucleus, as described previously, with a rounded-corners square sled, can fit in this configuration.

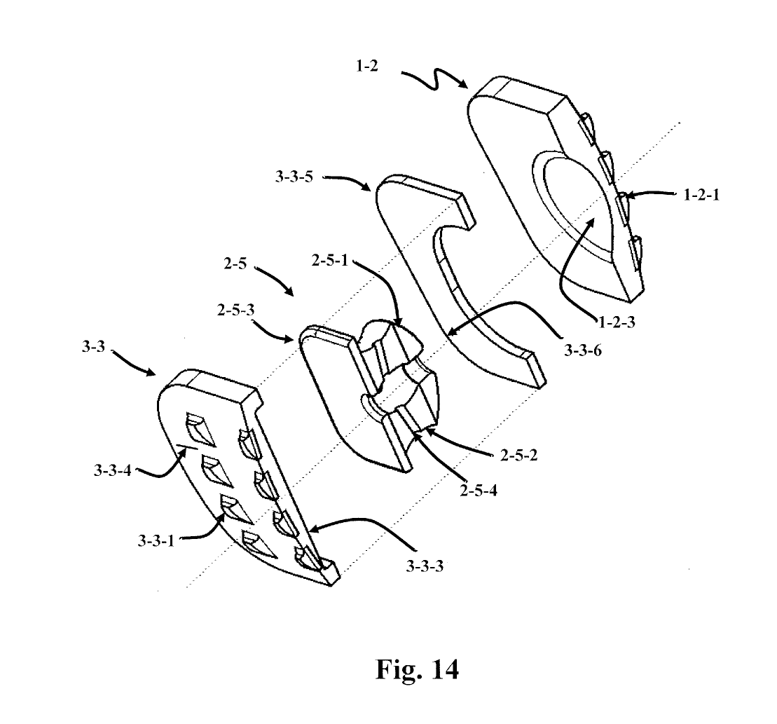

FIG. 14 shows an exploded, sagittal cutaway view of the embodiment seen in FIG. 10, and indicates how the planar surface mating of the lower surface of the nucleus sled 2-5-3 with the planar floor of the bottom endplate cavity 3-3-3 can establish a two-independent translational degrees-of-freedom planar joint in the z-axis and x-axis directions. In this embodiment, spherical cap 2-5-1 mates with conforming cavity 1-2-3 to form a ball-and-socket type joint with three independent degrees of rotational freedoms, wherein compression of the viscoelastic core 2-5-2 allows a third translational degree of freedom along the y-axis.

FIG. 15 and FIG. 16 depict two embodiments of nuclei with a round sled 2-6 and an rounded-corners square sled 2-7 without hydraulic portals, which can be substituted for nuclei embodiments 2-1 and 2-2 within invention embodiments of FIG. 1 and FIG. 10, respectively. These nuclei can be tripartite, graded, or a single polymer, as can the other embodiments discussed herein. Thus, the spherical cap 2-6-1 or 2-7-1, the core 2-6-2 or 2-7-2, and the sled 2-6-3 or 2-7-3 can be the same or different densities or materials. All the nuclei can provide some shock absorption and motion along the y-axis.

FIG. 17 shows embodiments of a sled 2-9-3 that press-fits or snap fits through the orifice 3-4-6 of cavity 3-4-3 with lip 3-4-5 integrated as part of bottom endplate 3-4. Once past the lip, the sled is free to translate and axially rotate in the plane of cavity 3-4-3. As with other endplates, features 3-4-1 can retain the plate within the intervertebral space by engaging bone, and surface 3-4-2 can be treated in a number of ways, such as, for example, plasma sintering or electron beam manufacture of a trabecular mesh structure, to encourage bone fusion. While this sled embodiment has a hydraulic portal, still other embodiments can have none

It can be seen that, in general, the invention comprises two plates, whose outer surface, in one embodiment, can be configured with keels, spikes, chevrons, or similar such structures. Alternatively, trabecular mesh treatment of those same surfaces can be used, all for fixing the endplates to bone and encouraging osteo-integration. The endplates can be separated by any one of a variety of nucleus embodiments that lock into the bottom plate and create a ball-and-socket joint with the top endplate and a planar joint with the bottom endplate. The various nuclei embodiments of the invention can be unified, integrated, graded, or tripartite polymer nuclei. They can also be configured with or without hydraulic portals and channels and with or without tubular extensions into the core body.

Certain embodiments of the invention, as seen in the figures, comprise two endplates: a top endplate 1-1 or 1-2, for example; a bottom endplate 3-1, 3-2, 3-3 or 3-4, for example; and a nucleus 2-n, n=1 to 7, for example. Nucleus sled 2-n-3, where n=1 to 7, can move planarly upon a horizontal floor (i.e., parallel to z-axis and x-axis plane of F₀) of a cavity in the superior surface of a bottom endplate to form a planar joint. The cavity can be circular, as shown by 3-1-3 or 3-2-3, for disc sleds, or rectangular, as shown by 3-3-3 or 3-4-3, for rectangular sleds. Lip 3-1-5 or 3-4-5 or retaining ring 3-2-5 or 3-3-5 can keep the sled and nucleus from being pulled out of a bottom endplate cavity. Spherical cap 2-n-1, wherein n=1 to 7, on the superior surface of nucleus 2-n, where n=1 to 7, conforms to spherical cavity 1-1-3 or 1-2-3 in the inferior surface of top endplate 1-1 or 1-2 to form a spherical joint.

After implanting an embodiment of the invention in an FSU or spinal joint, outer surface features on the endplates can hold the implant in place and any of a variety of surface treatments can assist in fusing those endplate surfaces to vertebral bone. For example, the endplate outer surfaces 1-1-4, 1-2-4, 3-1-4, 3-2-4, 3-3-4 and 3-4-4 can be treated with titanium plasma sprays or sintering, or one can generate a trabecular type titanium mesh employing Electron Beam Manufacturing. This allows the superior surface 1-1-4 or 1-2-4, of a top endplate to fuse to the superior vertebra and the inferior surface 3-1-4, 3-2-4, 3-3-4 and 3-4-4 of a bottom endplate to fuse to the inferior vertebra of a spinal joint. When spinal muscles dictate complex motion of a superior vertebra of a spinal joint, the top endplate of the embodiment can follow the same motion trajectory as the superior vertebra, since they are fused together. An embodiment of the invention can accommodate the resulting motion of the top endplate by means of a spherical joint, a planar joint and a flexible viscoelastic core.

In one embodiment, a spherical joint can provide up to three independent degrees of rotational freedom about the frontal, sagittal and central axes; a planar joint can provide up to two independent translational degrees of freedom along the frontal and sagittal axes; and the viscoelastic core can provide at least one degree of translational freedom along the central axis of the device. Since these axes are typically perpendicular to each other, they can be linearly independent. Vertebral motions along each axis within the limited range dictated by the spinal muscles, therefore, can be realized by an embodiment of the invention with six degrees of freedom and the range of motion prescribed by spinal kinematics.

In a further embodiment, nuclei 2-n, where n=1 to 7, is a flexible material, such as, for example, viscoelastic plastics, which can also add an infinite variety of motions due to the flexure in the material. In this sense, the invention encompasses the advantage of articulating joints to realize large motions and a viscoelastic core to realize finer motions of compression, extension, shear, torsion, or arbitrary combinations thereof. This joint action incorporates advantages of articulating joints with flexure joints and can be called a hybrid joint mechanism.

In an embodiment, top endplate 1-1 (or 1-2), fused with a superior vertebra and moving in concert with that vertebra, can rotate up to approximately ±10° about an axis comprising a linear combination of frontal (z-axis) and sagittal (x-axis) motions, while simultaneously performing unconstrained axial (y-axis) rotation. A spherical joint formed by nucleus spherical cap 2-n-1, where n=1 to 7, and conforming spherical cavity 1-1-3 (or 1-2-3) in the top endplate's inferior surface can produce these complex, coupled, motions. The independence of the joint rotations means the invention can allow, within joint limits incorporated in the design of the device, complex, coupled rotations normally dictated by the spinal muscles. Flexure of the core can also assist in such motions.

In an embodiment, as top endplate 1-1 (or 1-2) attempts to translate in the horizontal plane, forces between the spherical cavity 1-1-3 (or 1-2-3) and spherical cap 2-n-1, where n=1 to 7, cause the nucleus to slide on its sled in a horizontal plane (z-xis and x-axis plane parallel to F₀ z-axis and x-axis plane) and possibly compress or extend the nucleus's viscoelastic core along the central axis (y-axis). At the end of the motion, the top endplate, and the superior vertebra fused to it, will have translated and rotated to the required end position and orientation dictated by the spinal muscles.

If top endplate 1-1 (or 1-2) undergoes additional physiologic load, embodiments of the viscoelastic nucleus 2-n, where n=1 to 7, can compress along the central axis of the invention and can compress, extend, twist and bend under the influence of forces and moments-of-force. For certain embodiments, this can be normal operation. When physiologic load reduces on the top endplate during extension, a viscoelastic core can expand up to a non-loaded state, keeping the spherical cap and top endplate spherical cavity in continued contact until that point. Ideally, the expansion capabilities of the viscoelastic nucleus are such that the spherical end cap will maintain contact with the top endplate, even at maximum possible vertebral extension. Thus, at no time will the spherical cap be out of contact with the top end plate.

All displacements and rotations of the joints of the subject invention can be mechanically programmed to specific joint limits by appropriately installed joint stops. The joint stops can be rigid, or, to reduce wear, cushioned with materials falling within a wide range of durometer choices, from between approximately 50 to approximately 100. U.S. Pat. No. 8,277,505 teaches spherical joint stops that can be utilized with the spherical end cap and top end plate of the subject invention. U.S. Pat. No. 8,277,505 is hereby incorporated by reference for such teaching.

As further taught by U.S. Pat. No. 8,277,505, the use of spherical joint stops or sliders can keep the nucleus and the top endplate kinematically connected even during extension, since the spherical joint stops and/or sliders generate a profile-closed spherical joint of the spherical pair between the top endplate and the nucleus cap. In such an embodiment, the invention establishes a kinematic chain from bottom endplate to the top endplate, and, if a portion of the nucleus is compliant, a hybrid kinematic chain.

In one embodiment, a boot affixes to and extends between a top endplate 1-1 or 1-2 and a bottom endplate 3-1, 3-2, 3-3 or 3-4. In a more particular embodiment, a protective, fluids/gases impervious, tough, flexible, fiber-reinforced boot fixedly attaches to and between the two endplates. The boot can be designed to be under slight tension in the FSU's neutral position and, thus, oppose increasing extension of the nucleus embodiments described herein. Boot tension also can resist torsion loads on the device. In one embodiment, the boot utilizes a diamond weave fiber matrix. In another embodiment, the boot comprises tough diagonal fibers woven within the boot. The inventor's U.S. Pat. Nos. 7,927,375; 7,361,192 and 7,799,080, 8,226,724, all teach various boot embodiments that can be utilized with embodiments of the subject invention. These patents are hereby incorporated by reference herein, for such teachings, including any figures, tables, or drawings pertaining thereto.

Many previous spinal implants have been unable to imitate the full six-degrees of motion provided by a normal FSU within its workspace. Advantageously, the embodiments disclosed herein are able to provide up to six-degrees of rotational and translational movement, and can also provide profile-closure of at least one or more lower pair joints, for example, the planar and/or spherical pairs. Profile-closed joints help prevent improper or excessive motion between components. The embodiments described herein present a simple, but effective, fully motion capable, spinal implant wherein the nucleus, under normal operation, cannot be withdrawn from its bottom endplate cavity, yet all the while allowing the planar joint to function. With rotational sliders/joint-stops, the top endplate will not separate from the spherical cap of the nucleus without excessive external separation forces, yet all the while allowing the spherical joint to function.

Rotational sliders/joint-stops, as instructed in the inventor's U.S. Pat. No. 8,277,505, can be added to kinematically connect top endplate 1-1 or 1-2 to any spherical cap 2-n-1, where n=1-7, to profile-close the spherical joint (not shown here, but illustrated and instructed in the referenced patent) and simultaneously provide joint stops. Rotational sliders/joint-stops keep the top plate and spherical cap spherical surfaces in contact (profile-closed), even without external loads and, can prevent those circumstances wherein the top endplate spherical surface can otherwise ride upon the cap spherical surface, separating the COR of each and producing high wear curvate line contact between the articulating elements.

I claim:

1. A device comprising:
a nucleus having a cranial end and a caudal end, wherein the cranial end comprises a spherical cap and the caudal end comprises a sled and a core extending therebetween, the nucleus comprising, at least partially, a compliant material that provides the nucleus with at least one degree of axial freedom and a plurality of motion modalities about any axis of the device;
a top endplate comprising a superior surface and a spherical cavity opposite to the superior surface, wherein the compliant material of the nucleus maintains the spherical cap in constant slidable contact with the spherical cavity; and
a bottom endplate comprising an inferior surface and an endplate cavity and cavity lip opposite to the inferior surface, wherein the sled is slidably disposed within the endplate cavity and the cavity lip inhibits removal of the sled from the endplate cavity and the sled provides at least one degree of translational freedom;
such that the device forms a mechanical linkage of connected, articulating, and flexure components that provide at least one, and up to three, independent translational degrees of freedom, and at least one, and up to three, independent degrees of rotational freedom.

2. The device, according to claim 1, wherein the compliant material is a viscoelastic material.

3. The device, according to claim 1, wherein the nucleus comprises a single, unified construction.

4. The device, according to claim 1, wherein the nucleus comprises a tripartite construction, such that the spherical cap, sled, and core are fitted together.

5. The device, according to claim 2, wherein the spherical cap, sled, and core comprise two or more materials.

6. The device, according to claim 1, wherein the nucleus comprises variable densities.

7. The device, according to claim 6, wherein the nucleus comprises a transition region between the spherical cap and core and between the core and the sled.

8. The device, according to claim 7, wherein the transition regions comprise a mixture of the materials comprising the spherical cap, core, and sled.

9. The device, according to claim 4, further comprising one or more tubular projections on at least one of the spherical cap and sled for fitting them to the core.

10. The device according to claim 1, wherein the shape of the perimeter of the sled permits at least one degree of rotation of the sled within the endplate cavity.

11. The device according to claim 1, wherein the shape of the perimeter of the sled permits simultaneously two degrees of translation and one of rotation.

12. The device, according to claim 1, wherein the shape of the sled perimeter is a circle, an oval, a rounded-corner square, or a rectangle.

13. The device, according to claim 1, wherein the shape of the sled perimeter is a convex planar curve.

14. The device, according to claim 13, wherein the shape of the perimeter of the endplate cavity at least generally corresponds to the shape of the perimeter of the sled.

15. The device, according to claim 14, wherein the slide slides within the endplate cavity up to approximately 1.0 mm in any direction perpendicular to a vertical axis of the device.

16. The device, according to claim 1, further comprising a retention ring to secure the sled in the endplate cavity, wherein the cavity lip is part of the retention ring.

17. The device, according to claim 1, further comprising a flexible boot, comprising a fluids and gases impervious material, fixedly attached to the top endplate and the bottom end plate.

18. The device, according to claim 17, wherein the boot material is fiber-reinforced.

19. The device, according to claim 1, wherein at least one of the top endplate and bottom end plate comprise one or more securing structures for bone ingrowth.

20. The device, according to claim 13, wherein the nucleus further comprises a central chamber.

21. The device, according to claim 20, further comprising one or more hydraulic portals contiguous with the central chamber.

22. A device comprising:
a nucleus having a cranial end and a caudal end, the nucleus comprising, at least partially, a compliant material that provides the nucleus with at least one degree of axial freedom and a plurality of motion modalities about any axis of the device;
a top endplate comprising a superior surface and a spherical cavity opposite to the superior surface, wherein the cranial end of the nucleus maintains constant slidable contact with the spherical cavity; and
a bottom endplate comprising an inferior surface and an endplate cavity and cavity lip opposite to the inferior surface, wherein the caudal end of the nucleus is slidably disposed within the endplate cavity and the cavity lip inhibits removal of the caudal end from the endplate cavity and wherein the caudal end of the nucleus and the endplate cavity provide at least one degree of translational freedom;
such that the device forms a mechanical linkage of connected, articulating, and flexure components that provide at least one, and up to three, independent translational degrees of freedom, and at least one, and up to three, independent degrees of rotational freedom.

23. The device, according to claim 22, wherein at least a portion of the nucleus comprises a viscoelastic material.

24. The device, according to claim 22, wherein the nucleus comprises two or more materials.

25. A device comprising:
a nucleus having a cranial end and a caudal end, wherein the cranial end comprises a spherical cap and the caudal end comprises a sled and a core extending therebetween, wherein the nucleus provides at least one degree of axial freedom;
a top endplate comprising a superior surface and a spherical cavity opposite to the superior surface, wherein the spherical cap maintains constant slidable contact with the spherical cavity; and
a bottom endplate comprising an inferior surface and an endplate cavity and cavity lip opposite to the inferior surface, wherein the sled is slidably disposed within the endplate cavity and the cavity lip inhibits removal of the sled from the endplate cavity and wherein the sled provides at least one degree of translational freedom;
such that the device forms a mechanical linkage of connected, articulating, and flexure components that provide at least one, and up to three, independent translational degrees of freedom, and at least one, and up to three, independent degrees of rotational freedom.
